(19) United States
(12) Patent Application Publication
(10) Pub. No.: US 2007/0265469 A1
(43) Pub. Date: Nov. 15, 2007

(54) Fatty Acid Esters of Alkanolamines and Their Use as Softening Agents

(76) Inventors: John Stuart Cowman, Bradford (GB); Adrian Fox, Normanton (GB); Paul Dekock, Bradford (GB)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/665,439

(22) PCT Filed: Oct. 12, 2005
(86) PCT No.: PCT/EP05/55200

§ 371 Date: Jul. 24, 2007

(30) Foreign Application Priority Data
Oct. 13, 2004 (EP) 04024325.5

(51) Int. Cl. C07C 219/06; C07C 213/06; C07C 219/08; C11D 1/46; C11D 1/62; C11D 3/30

(52) U.S. Cl. 562/561

(57) ABSTRACT

The instant invention relates to the use of compounds derived from the esterification reaction of alkoxylated amines and fatty acids, optionally quaternised with an alkylating agent, and of the cationic surfactants and esterquats obtainable therefrom, as softening agents in the textile and paper industry and in the field of personal care.

The instant invention relates to the use of compounds derived from the esterification reaction of alkoxylated amines and fatty acids, optionally quaternised with an alkylating agent, and of the cationic surfactants and esterquats obtainable therefrom, as softening, debonding and bulking agents for natural and synthetic fibres, employed in the pulp and paper industry.

The cationic surfactants and partial esterquats thus obtained exhibit a high degree of efficacy in softening and conditioning natural and synthetic fibres. This chemistry may be used for softening, bulking or debonding applications in the pulp and paper industry.

### PRIOR ART

Cationic surfactants derived from amines have been widely used for some decades as softening, debonding and conditioning agents for natural and synthetic fibres of all types, and are used in fields such as the treatment of textile fibres and of paper and in hair care products.

Owing to their greater biodegradability, the use of cationic amine derivatives in which the hydrophobic hydrocarbon chains are interrupted by functional ester groups has been employed for several years, those used mostly being the quaternized derivatives of polyalkanolamine esters, generally known as “esterquats”. Typically, the types used most are esterquats derived from triethanolamine, owing to their lower cost.

It is also well known that the above-mentioned esterquats are prepared from alkanolamine esters, produced previously by an esterification reaction of the alkanolamine with fatty acids or functionalized reactive derivatives thereof, by their quaternization with alkylation agents such as alkyl halides or sulphates. There is an abundant bibliography on the subject, amongst which FR-A-1593921, EP-A-239910, EP-A-295385, WO-A-9101295, DE-A-19539846 and WO-A-9849132, amongst many others, may be mentioned.

However, it is well known that esterquats are less effective softeners than their homologues which do not contain ester groups, and this has led to various technical developments directed towards improving the softening efficacy of these esterquats.

British patent GB 866408 describes novel quaternary ammonium compounds for treating textile materials, in which the nitrogen atoms in the alkanolamine structure are attached to hydroxyalkyl groups, with no provision for polyalkoxylated variants.

U.S. Pat. No. 4,439,331 mentions a liquid textile softener manufactured from esterified polyalkoxylated diamines, in which the tertiary nitrogen groups are fully quaternised with an alkylating agent. Isopropanol is employed as a solvent in the quaternising procedure, to lower an unmanageably high viscosity, and a nonionic dispersant is preferentially added to improve the dispersability of the product in water.

Thus, U.S. Pat. No. 5,593,614 describes the improvement of the softening effect of esterquats by mixing them with non-ionic surfactants, U.S. Pat. No. 5,501,806 proposes the mixing of esterquats with other cationic surfactants, and EP-A-394133 describes the use of acrylic cationic polymers as additives for improving softness.

British patent GB-602048 describes oligomeric alkanolamine esters based on the esterification reaction of triethanolamine with dicarboxylic acids and fatty acids, as well as their quaternization with methyl chloride or dimethyl sulphate and their use as softening agents for natural and synthetic fibres, and U.S. Pat. No. 4,719,382 and U.S. Pat. No. 4,237,016 describe the use of the esterquats described in the above-mentioned British patent, amongst cationic polymers of many other types, as additives for improving the softening efficacy of cationic surfactants which do not contain ester groups. Moreover, WO-A-9812293 describes the use of the same oligomeric esterquats as additives for incorporation in the aqueous phase of softening compositions, which contain esterquats with the object of improving their softening efficacy.

German patent DE-19539846 describes the synthesis of esterquats derived from dicarboxylic acids, fatty acids and triethanolamine and their use as hair conditioners, and patent DE-19715835 describes esterquats based on the reaction of methyl diethanolamine and mixtures of fatty acids and dicarboxylic acids, with subsequent ethoxylation and/or quaternization.

WO-A-9849132 describes the synthesis of esterquats derived from dicarboxylic acid/fatty acid/triethanolamine, within a specific selected range of proportions, and their use in fabric-softening compositions.

Lastly, patent DE-19519876 describes esterquats based on the reaction of a trialkanolamine with mixtures of fatty acids, dicarboxylic acids, and sorbitol and the subsequent quaternization and/or ethoxylation of the esters produced.

However, as far as the authors of the present invention know, the prior art relating to quaternised esters does not allow all the desired properties of a paper softener to be achieved through the use of a single compound. The paper industry has unique requirements for softener chemicals and among those desired properties are affinity for paper fibres, good softening effect, rewettability and increased bulk of the paper sheet, ready biodegradability of a liquid, low viscosity product. The present invention describes how such properties may be optimised through modification of the chemistry and realised in a single molecule.

### SUMMARY OF THE INVENTION

The subject of the present invention is the use of esters derived from alkanolamines, fatty acids and alkylating agents and of the cationic surfactants obtainable therefrom in the pulp and paper industry.

Also included within the subject of the present invention is the use of the cationic surfactants based on the said esters derived from alkanolamines and fatty acids, particularly the partial esterquats obtainable therefrom, as conditioning and softening agents for natural and synthetic fibres.

### DESCRIPTION OF THE INVENTION

The alkanolamine esters used in the instant invention are obtained by the esterification reaction of an alkanolamine of general formula (I) 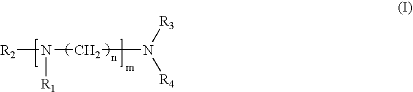
with a carboxylic acid or a reactive derivative thereof, of general formula R₅COOH, or with a dicarboxylic acid or a reactive derivative thereof, of general formula HOOC—R₆—COOH,
in which at least one of the radicals R₁ to R₄, preferably each of them, is —[CH₂CHR₇O]ₚ—H, the others being H or C₁-C₆ alkyl, R₅ is a linear or branched C₂-C₂₂-alkyl or -alkenyl group, R₆ is a C₁-C₃₆-alkylene group, optionally substituted or unsaturated, or an arylene group, R₇ is H or C₁-C₄-alkyl, n is a number between 1 and 20, m is a number between 1 and 5 and p is a number between 1 and 10. Examples of reactive derivatives of the carboxylic acids are their esters, their anhydrides or their acid chlorides.

Within the present invention, the degree of esterification, the length of alkyl chain of the carboxylic acid and the level of saturation/unsaturation within the alkyl chain can be used to modify the softening/debonding performance. It is well known that longer chain (C₁₈₋₂₂) acids with a high level of saturation provide the best softening performance of a paper sheet. It is also known that such long chain saturated carboxylic acids contribute to unfavourable properties such as water repellency (poor rewetting) and solid or paste-like products, which are not easy to handle or dispense in a paper mill. It has now been discovered that a triester of an alkoxylated diamine, in which the carboxylic acid is based on a C₁₈-alkenyl chain, having additionally been partially alkylated to form a partially quaternised cationic surfactant, provides a liquid product with good softening and rewetting properties.

Examples of alkanolamines are the reaction products of aliphatic diamines, triamines or polyamines with alkylene oxides, in which each hydrogen on the amine nitrogen atoms is replaced with a minimum of one mole alkylene oxide. Typically, the reaction product of ethylene diamine with more than four moles ethylene oxide has proven effective.

It has been discovered within the present invention that the degree of alkoxylation of the di- or polyamine, in the cationic surfactant, can be controlled to provide optimum rewetting properties for applications in the pulp and paper industry.

Therefore preferred is the use of alkanolamine esters wherein reactive derivatives of the carboxylic acids are their esters, their anhydrides or their acid chlorides, and wherein
R₁ to R₄ are —[CH₂CHR₇O]ₚ—H,
n is 1 to 4,
m is 1 to 3,
p is 1 to 5,
R₅ is a linear or branched C₁₂-C₂₀-alkyl or -alkenyl group,
R₆ is a C₂₄-C₃₆-alkylene group, and
R₇ is H or methyl.

Especially preferred is the use of alkanolamine esters wherein
R₁ to R₄ are —[CH₂CHR₇O]ₚ—H,
n is 2,
m is 1,
p is 1 to 3,
R₅ is a linear or branched C₁₂-C₂₀-alkenyl group, and
R₇ is H.

Examples of fatty acids which may be included in the esterification reaction are those obtained from vegetable and animal oils and fats such as those obtained from coconut, tallow, palm, sunflower, soya, olein, oil greaves, etc., optionally wholly or partially hydrogenated, as well as purified or synthetic fatty acids such as lauric, stearic, palmitic, oleic, linoleic, and 2-ethylhexanoic acids, etc.

The molar ratio of the carboxylic acid to the alkanolamine is between 0.5 and 3.5, preferably between 1.0 and 3.5, most preferably between 2.5 and 3.5.

The esterification reaction is preferably performed by condensation of the fatty acid, with a mixture of the alkanolamine, at a temperature of between 120° C. and 220° C., for a period of from 2 to 10 hours, preferably at a reduced pressure of about 5 to 200 mbar and in the presence of some of the catalysts already known for the esterification of conventional esterquats, for example, hypophosphorous acid and paratoluene sulphonic acid, and also in the presence of some of the usual stabilizers and antioxidants such as tocopherols, BHT, BHA, citric acid, etc. It will be clear to a person skilled in the art that the esterification reaction may alternatively also be performed by other conventional techniques starting with reactive derivatives of the carboxylic acids, for example, their esters, their anhydrides, or their acid chlorides.

The esters thus produced are useful for preparing cationic surfactants efficacious for use in the softening, bulking and debonding treatment of natural and synthetic fibres such as those used in the pulp and paper industry. The cationic surfactants may be the esterquats obtainable by their quaternization with alkylation agents, or addition salts of the alkanolamine esters of the invention with mineral or organic acids such as hydrochloric, sulphuric, phosphoric, citric, and lactic acids, etc. The partial esterquats, in which some nitrogen atoms within the molecule are quaternised and others remain as tertiary amine salts, are preferred as cationic fibre-softening surfactants.

The partial esterquats are produced from the alkanolamine esters of the invention by an additional quaternization reaction, also known per se, for example, as described in the above-mentioned patent application WO-A-9849132.

For example, the reaction mixture resulting from the esterification is reacted with alkylating products such as methyl chloride, benzyl chloride, methyl bromide, dimethyl sulphate, diethyl sulphate, dimethyl carbonate, etc., optionally in the presence of organic solvents which facilitate the handling thereof, such as propylene glycol, ethylene glycol, dipropylene glycol, etc., and the pH is subsequently adjusted to between 1.5 and 7.0, preferably between 2 and 4.5 by the addition of an acid such as any of hydrochloric, sulphuric, phosphoric, citric acids, etc.

Preferred alkylating agents are methyl chloride, benzyl chloride and dimethyl sulfate, the most preferred being dimethyl sulfate. The degree of alkylation is critical and is controlled to optimise cationic charge (under neutral pH conditions), product viscosity and dispersability of the final product in water. Fully quaternised esters provide too much water solubility, decreased softening efficiency and unmanageably high product viscosities and therefore partially quaternised alkanolamine esters are preferred.

Assuming all nitrogen atoms are in the form of tertiary amine, after esterification, the degree of alkylation would lie within the range 20 to 80% for each available nitrogen, referred to as partial quaternisation (i.e. 0.2 to 0.8 moles of alkylating agent for every mole of tertiary amine, the preferred range being 0.4-0.6).

The cationic surfactants obtainable from the alkanolamine esters of the invention may be used without further modification, as pre-prepared dispersions in water, with or without additional surfactants, or in blends with other additives such as hydroxy compounds. Typical examples of other additives are alkylene glycols, polyalkylene glycols, glycerols and polyglycerols, sugars such as sorbitol, glucose and fructose.

The cationic surfactants obtainable from the alkanolamine esters of the invention exhibit a high degree of fibre-softening efficacy and, moreover, owing to their degree of biodegradability, are very well tolerated from the ecological point of view. Moreover, even if the said surfactants are not used in a major or predominant proportion, they considerably improve the softening efficacy of compositions based on conventional esterquats and other cationic surfactants and, when used as fabric softeners, counteract the adverse effect of the presence of anionic surfactant residues in the textile fibres after washing and during the rinsing stage.

In summary, the cationic surfactants of the present invention have been optimised to provide the most beneficial properties for the pulp and paper industry. At each reaction stage, raw material selection and molecular ratios were carefully selected. Choice of amine, degree of alkoxylation, fatty acid selection, degree of esterification and alkylation are all influential in controlling the properties of the final product.

The cationic surfactants obtainable from the partially quaternised alkanolamine esters of the invention are intended for use in the pulp and paper industry for the softening of tissue or as a debonding agent for fluff pulps. The softening and debonding properties also increase the bulk (decrease the density) of a paper sheet, allowing its use as a bulking agent for printing and writing grades of paper. For the tissue industry, rewettability (a measure of water adsorption) of the paper sheet is of paramount importance, since it is known that some products from the prior art are known to confer a degree of water repellency to the substrate. The cationic surfactants of this invention display excellent rewetting tendencies.

With the products defined in this patent application, particularly those in which the fatty chains have unsaturated bonds, it is possible to produce softening formulations which are translucent or transparent without the need to use the solvents or emulsifying agents usually used for formulas of this type.

The following examples shall explain the instant invention in more detail.

### EXAMPLES

### Example 1

### (Refers to Prior Art)

Oleic acid (560 g) and triethanolamine (149 g) were mixed in a reaction flask equipped with a stirrer, a temperature probe and an inlet for an inert gas. 50% by weight hypophosphorous acid (1.4 g) was then added with stirring. The mixture was heated to 170° C., under a constant stream of nitrogen gas, and this temperature was maintained whilst the esterification water was distilled and until the acid value of the mixture was below 5 mg KOH/g. The mixture was then cooled to 70° C. and isopropyl alcohol (70 g) added. The temperature was adjusted to 60° C. and dimethyl sulphate (119 g) added slowly over 6 hours, keeping the temperature between 60 and 65° C. Finally the mixture was cooled to 50° C. The yield of finished softener concentrate was 865 g.

### Example 2

Vegetable fatty acid (362.3 g) and the reaction product of ethylene diamine with 6 moles ethylene oxide (alkanolamine, 129.5 g) were mixed in a reaction flask equipped with a stirrer, a temperature probe and an inlet for an inert gas. 50% by weight hypophosphorous acid (0.8 g) was then added with stirring. The mixture was heated to 170° C., under a constant stream of nitrogen gas, and this temperature was maintained whilst the esterification water was distilled and until the acid value of the mixture was below 5 mg KOH/g. The mixture was then cooled to 70° C. and dipropylene glycol (25.4 g) added. The temperature was adjusted to 60° C. and dimethyl sulphate (65.3 g) added slowly over 6 hours, keeping the temperature between 60 and 65° C. Finally the mixture was cooled to 50° C. and dipropylene glycol (25.4 g) added. The yield of finished softener concentrate was 600.0 g.

Using the procedure in Example 2, several variations of this ester-quat chemistry were produced, using different raw materials and molecular ratios. These variations are summarised in the table below (Examples 3-32)

TABLE 1
EXAMPLES 3-32

| Ex. | Alkanolamine | Fatty acid | Quaternisation |
|---|---|---|---|
| 3 | Ethylene diamine + 6 | Oleic acid (2.0 moles) | Dimethyl sulphate |
| | E.O. (1 mole) | | (1 mole) |
| 4 | Ethylene diamine + 6 | Oleic acid (2.5 moles) | Dimethyl sulphate |
| | E.O. (1 mole) | | (1 mole) |
| 5 | Ethylene diamine + 6 | Oleic acid (3.0 moles) | Dimethyl sulphate |
| | E.O. (1 mole) | | (1 mole) |
| 6 | Ethylene diamine + 6 | Tallow fatty acid | Dimethyl sulphate |
| | E.O. (1 mole) | (2.0 moles) | (1 mole) |
| 7 | Ethylene diamine + 6 | Tallow fatty acid | Dimethyl sulphate |
| | E.O. (1 mole) | (2.5 moles) | (1 mole) |
| 8 | Ethylene diamine + 6 | Tallow fatty acid | Dimethyl sulphate |
| | E.O. (1 mole) | (3.0 moles) | (1 mole) |
| 9 | Ethylene diamine + 6 | Vegetable fatty acid | Dimethyl sulphate |
| | E.O. (1 mole) | (2.0 moles) | (1 mole) |
| 10 | Ethylene diamine + 6 | Vegetable fatty acid | Dimethyl sulphate |
| | E.O. (1 mole) | (2.5 moles) | (1 mole) |
| 11 | Ethylene diamine + 6 | Vegetable fatty acid | Dimethyl sulphate |
| | E.O. (1 mole) | (3.0 moles) | (1 mole) |
| 12 | Ethylene diamine + 8 | Oleic acid (2.0 moles) | Dimethyl sulphate |
| | E.O. (1 mole) | | (1 mole) |
| 13 | Ethylene diamine + 8 | Oleic acid (2.5 moles) | Dimethyl sulphate |
| | E.O. (1 mole) | | (1 mole) |
| 14 | Ethylene diamine + 8 | Oleic acid (3.0 moles) | Dimethyl sulphate |
| | E.O. (1 mole) | | (1 mole) |
| 15 | Ethylene diamine + 8 | Tallow fatty acid | Dimethyl sulphate |
| | E.O. (1 mole) | (2.0 moles) | (1 mole) |
| 16 | Ethylene diamine + 8 | Tallow fatty acid | Dimethyl sulphate |
| | E.O. (1 mole) | (2.5 moles) | (1 mole) |
| 17 | Ethylene diamine + 8 | Tallow fatty acid | Dimethyl sulphate |
| | E.O. (1 mole) | (3.0 moles) | (1 mole) |
| 18 | Ethylene diamine + 8 | Vegetable fatty acid | Dimethyl sulphate |
| | E.O. (1 mole) | (2.0 moles) | (1 mole) |
| 19 | Ethylene diamine + 8 | Vegetable fatty acid | Dimethyl sulphate |
| | E.O. (1 mole) | (2.5 moles) | (1 mole) |
| 20 | Ethylene diamine + 8 | Vegetable fatty acid | Dimethyl sulphate |
| | E.O. (1 mole) | (3.0 moles) | (1 mole) |
| 21 | Ethylene diamine + 12 | Oleic acid (2.0 moles) | Dimethyl sulphate |
| | E.O. (1 mole) | | (1 mole) |
| 22 | Ethylene diamine + 12 | Oleic acid (2.5 moles) | Dimethyl sulphate |
| | E.O. (1 mole) | | (1 mole) |
| 23 | Ethylene diamine + 12 | Oleic acid (3.0 moles) | Dimethyl sulphate |
| | E.O. (1 mole) | | (1 mole) |
| 24 | Ethylene diamine + 12 | Tallow fatty acid | Dimethyl sulphate |
| | E.O. (1 mole) | (2.0 moles) | (1 mole) |
| 25 | Ethylene diamine + 12 | Tallow fatty acid | Dimethyl sulphate |
| | E.O. (1 mole) | (2.5 moles) | (1 mole) |
| 26 | Ethylene diamine + 12 | Tallow fatty acid | Dimethyl sulphate |
| | E.O. (1 mole) | (3.0 moles) | (1 mole) |
| 27 | Ethylene diamine + 12 | Vegetable fatty acid | Dimethyl sulphate |
| | E.O. (1 mole) | (2.0 moles) | (1 mole) |
| 28 | Ethylene diamine + 12 | Vegetable fatty acid | Dimethyl sulphate |
| | E.O. (1 mole) | (2.5 moles) | (1 mole) |
| 29 | Ethylene diamine + 12 | Vegetable fatty acid | Dimethyl sulphate |
| | E.O. (1 mole) | (3.0 moles) | (1 mole) |
| 30 | Ethylene diamine + 6 | Vegetable fatty acid | Dimethyl sulphate |
| | E.O. (1 mole) | (3.0 moles) | (1.25 mole) |
| 31 | Ethylene diamine + 6 | Vegetable fatty acid | Dimethyl sulphate |
| | E.O. (1 mole) | (3.0 moles) | (1.5 mole) |
| 32 | Ethylene diamine + 6 | Vegetable fatty acid | Dimethyl sulphate |
| | E.O. (1 mole) | (3.0 moles) | (1.75 mole) |

### EVALUATION OF CHEMISTRY FOR THE PAPER INDUSTRY

The samples produced from examples 1 and 3-32 were assessed in a papermaking laboratory, to evaluate their debonding and softening performance on a paper sheet. The paper tissue industry is one of several outlets for this new chemistry, which modifies inter-fibre bonding, increases bulk (reduces density) and provides a softer feel for tissue papers. Tissue grades of paper are very light in weight, for example 18 grams per square meter (gsm), and difficult to simulate in a laboratory. Heavier (100 gsm) sheets are produced in the laboratory and comparisons made with prior art, to evaluate performance. Two different techniques were used for evaluation. Wet-end application, where product is added to a slurry of paper fibres in water, followed by sheet making, drying and the measurement of physical properties of the paper sheet, and surface application, where a dilute dispersion of product in water is sprayed on to the surface of a paper sheet. After drying, the physical properties of the sheet are measured.

Wet End Application

1 litre of stock (paper fibre slurry) was placed in a suitable container along with the required softener addition and stirred at 500 rpm for 60 seconds. The addition level of each softener was adjusted to a value of 0.4% active material. 200 ml samples of the treated stock were then taken and formed into a handsheet using the British Standard Sheet Forming Apparatus. For each softener, 4 hand-sheets were made, to obtain a meaningful average. “Control” sheets contained no softener product. The sheets were then pressed onto stainless steel plates at 4.0 bar for 4 minutes, placed into drying rings and dried at 100° C. for 30 minutes. After conditioning at 50°RH and 23° C. for a minimum period of 12 hours the sheets were ready and the following tests were carried out:

Burst Testing

The sheets were subjected to dry burst strength testing (TAPPI Std T403 OM-91, Bursting Strength of Paper). Results were recorded as a burst index (=burst value in kPa divided by the sheet weight in grams per square meter)

Softness (Hand Feel)

This is purely a subjective test but is the industry standard. The comparative degree of softness is assessed against the control samples (no softener). The paper sheets were crumpled and then felt by hand to see if there was a noticeable difference in softness. Six people made up the panel of assessors, giving scores of 1 (no discernable softness) to 5 (excellent softness). The average results from six people were recorded.

Bulk

The sheets were weighed and the caliper (thickness) measured. Bulk is calculated by taking the caliper measurement in micrometers, divided by the sheet weight in grams per square meter.

Stiffness

The sheets are subject to stiffness testing using a L & W Stiffness Tester (TAPPI Std T556). Results were recorded as a Stiffness Index (=stiffness value in mN divided by the sheet weight in grams per square meter).

Wicking Test

This test looks at the rewettability of the sheets in comparison to that of the untreated sheet. Test strips, 15 mm wide, are hung vertically, side by side. The lowest 5 mm of the strips were in allowed to come into with water. After 30 minutes, the water was removed and the wet line (the distance, in mm, the water had traveled up the test strip).

Spray Application

1 litre of stock was placed in a suitable container and stirred at 500 rpm for 60 seconds. 200-ml samples of the untreated stock were then taken and formed into a hand-sheets using the British Standard Sheet Forming Apparatus. For each product under analysis, 4 untreated hand-sheets were produced. The control sheets were sprayed with water only. Prior to the spray application, the sheets were then pressed onto stainless steel plates at 4.0 bar for 4 minutes. After the pressing, the sheets are ready for spraying. Each product was made up at 0.2% (active material) and then sprayed at a distance of approximately 30 cm for 6 seconds. This gave a dry pick up of around 0.1 g/m². After each individual sheet had been sprayed, the sheet was weighed and the wet pick up calculated. The sheet was then placed into drying rings and dried at 100° C. for 30 minutes. After conditioning at 50°RH and 23° C. for a minimum period of 12 hours the sheets were ready for assessment: The sprayed sheets were evaluated according to the previously mentioned methods for burst index, softness (hand feel) and wicking.

TABLE 2
APPLICATION RESULTS

| | Wet-end addition | | | | | Surface addition | | |
|---|---|---|---|---|---|---|---|---|
| | | | | Softness | Wicking | | Softness | Wicking |
| | Burst | Bulk | Stiffness | (hand | Rewetability | Burst | (hand | Rewetability |
| Ex. | index | index | index | feel) | (mm) | index | feel) | (mm) |
| Control | 1.02 | 1.96 | 2.86 | 1.0 | 121 | 0.76 | 1.0 | 128 |
| 1 | 0.82 | 2.04 | 2.29 | 3.4 | 91 | 0.54 | 2.9 | 116 |
| 3 | 0.64 | 2.08 | 2.15 | 3.3 | 115 | 0.49 | 2.2 | 124 |
| 4 | 0.55 | 2.10 | 2.14 | 3.9 | 114 | 0.42 | 2.8 | 124 |
| 5 | 0.48 | 2.17 | 1.90 | 4.6 | 112 | 0.37 | 3.7 | 123 |
| 6 | 0.62 | 2.05 | 2.17 | 3.6 | 110 | 0.48 | 2.5 | 118 |
| 7 | 0.59 | 2.09 | 2.05 | 4.0 | 108 | 0.44 | 2.9 | 116 |
| 8 | 0.51 | 2.14 | 1.98 | 4.7 | 105 | 0.35 | 3.9 | 113 |
| 9 | 0.65 | 2.04 | 2.31 | 3.3 | 114 | 0.50 | 2.1 | 123 |
| 10 | 0.56 | 2.09 | 2.14 | 3.9 | 114 | 0.44 | 2.8 | 122 |
| 11 | 0.49 | 2.18 | 1.92 | 4.5 | 111 | 0.37 | 3.6 | 122 |
| 12 | 0.75 | 2.03 | 2.27 | 3.1 | 116 | 0.57 | 2.0 | 125 |
| 13 | 0.72 | 2.05 | 2.22 | 3.5 | 115 | 0.50 | 2.5 | 125 |
| 14 | 0.68 | 2.09 | 2.10 | 4.1 | 115 | 0.39 | 3.4 | 124 |
| 15 | 0.64 | 2.06 | 2.23 | 3.4 | 112 | 0.54 | 2.2 | 119 |
| 16 | 0.59 | 2.11 | 2.02 | 3.9 | 109 | 0.49 | 2.6 | 119 |
| 17 | 0.53 | 2.15 | 1.94 | 4.2 | 106 | 0.47 | 3.6 | 118 |
| 18 | 0.82 | 2.04 | 2.39 | 3.1 | 116 | 0.58 | 1.9 | 125 |
| 19 | 0.77 | 2.07 | 2.21 | 3.8 | 115 | 0.52 | 2.5 | 124 |
| 20 | 0.68 | 2.12 | 2.03 | 4.1 | 113 | 0.40 | 3.3 | 122 |
| 21 | 0.78 | 2.02 | 2.39 | 2.9 | 118 | 0.61 | 1.8 | 125 |
| 22 | 0.76 | 2.06 | 2.30 | 3.2 | 117 | 0.60 | 2.3 | 124 |
| 23 | 0.69 | 2.09 | 2.25 | 3.8 | 118 | 0.49 | 3.0 | 124 |
| 24 | 0.77 | 2.05 | 2.30 | 3.2 | 113 | 0.57 | 2.0 | 120 |
| 25 | 0.69 | 2.09 | 2.18 | 3.8 | 110 | 0.50 | 2.4 | 119 |
| 26 | 0.67 | 2.12 | 1.98 | 3.9 | 109 | 0.48 | 3.3 | 119 |
| 27 | 0.88 | 2.00 | 2.45 | 2.9 | 119 | 0.62 | 1.8 | 124 |
| 28 | 0.79 | 2.03 | 2.36 | 3.5 | 116 | 0.59 | 2.2 | 124 |
| 29 | 0.75 | 2.07 | 2.22 | 3.7 | 114 | 0.50 | 3.1 | 123 |
| 30 | 0.47 | 2.19 | 1.89 | 4.7 | 112 | 0.38 | 3.6 | 124 |
| 31 | 0.49 | 2.18 | 1.91 | 4.4 | 113 | 0.43 | 3.5 | 124 |
| 32 | 0.53 | 2.15 | 1.94 | 4.2 | 115 | 0.44 | 3.2 | 125 |

### INTERPRETATION OF RESULTS

Softening chemicals interfere with fibre-fibre bonding. This debonding effect influences the physical strength of the paper sheet. Softening performance can therefore be assessed by comparing burst and stiffness measurements (the lower the number, the better the performance) together with bulk, wicking and hand feel results (the higher the number the better the performance.

The Control samples, with no softener compounds, always record the highest burst index and stiffness index values. Softening and or debonding efficiency can be evaluated by comparing the reduction in strength or stiffness, achieved with instant compounds, versus the prior art of example 1.

1. A process for softening, debonding, or bulking pulp comprising the step of mixing at least one fatty acid ester compound wherein the at least one fatty acid ester compound is a product of an esterification reaction of an alkanolamine of general formula (I) 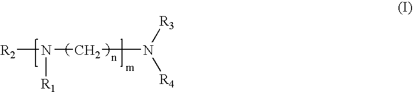
with a carboxylic acid or a reactive derivative thereof, of general formula
R₅COOH,
or with a dicarboxylic acid or a reactive derivative thereof, of general formula
HOOC—R₆—COOH,
wherein at least one of the radicals R₁ to R₄ is —[CH₂CHR₇O]ₚ—H, the others being H or C₁-C₆ alkyl,
R₅ is a linear or branched C₂-C₂₂-alkyl or -alkenyl group,
R₆ is a C₁-C₃₆-alkylene group, or an arylene group,
R₇ is H or C₁-C₄-alkyl,
n is a number between 1 and 20,
m is a number between 1 and 5, and
p is a number between 1 and 10,
with a pulp slurry.

2. A process according to claim 1 wherein reactive derivatives of the carboxylic acids are their esters, their anhydrides or their acid chlorides, and wherein
R₁ to R₄ are —[CH₂CHR₇O]ₚ—H,
n is 1 to 4,
m is 1 to 3,
p is 1 to 5,
R₅ is a linear or branched C₁₂-C₂₀-alkyl or -alkenyl group,
R₆ is a C₂₄-C₃₆-alkylene group, and
R₇ is H or methyl.

3. A process according to claim 1 wherein
R₁ to R₄ are —[CH₂CHR₇O]ₚ—H,
n is 2,
m is 1,
p is 1 to 3,
R₅ is a linear or branched C₁₂-C₂₀-alkenyl group, and R₇ is H.

4. A process according to claim 1 wherein the molar ratio of the carboxylic acid to the alkanolamine is between 0.5 and 3.5.

5. A process according to claim 1 wherein the at least one fatty acid ester compound is partially quaternised with an alkylating agent.

6. A process according to claim 5 wherein the at least one fatty acid ester compound is partially quaternised with methyl chloride, benzyl chloride, dimethyl sulfate or diethyl sulphate.

7. A process according to claim 1 wherein the at least one fatty acid ester compound is a pre-prepared dispersion in water or is in a blend with a hydroxy compound.

8. A process according to claim 1, wherein R₆ is a substituted or unsaturated C₁-C₃₆-alkylene group.

9. A process according to claim 1 wherein the molar ratio of the carboxylic acid to the alkanolamine is between 1.0 and 3.5.

10. A process according to claim 1 wherein the molar ratio of the carboxylic acid to the alkanolamine is between 2.5 and 3.5.

11. A process for softening, debonding, or bulking paper comprising the step of applying at least one fatty acid ester compound to a paper, wherein the at least one fatty acid ester compound is a product of an esterification reaction of an alkanolamine of general formula (I) 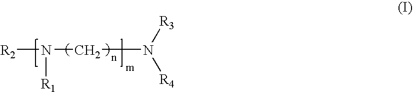
with a carboxylic acid or a reactive derivative thereof, of general formula
R₅COOH,
or with a dicarboxylic acid or a reactive derivative thereof, of general formula
HOOC—R₆—COOH,
wherein at least one of the radicals R₁ to R₄ is —[CH₂CHR₇O]ₚ—H, the others being H or C₁-C₆ alkyl,
R₅ is a linear or branched C₂-C₂₂-alkyl or -alkenyl group,
R₆ is a C₁-C₃₆-alkylene group, or an arylene group,
R₇ is H or C₁-C₄-alkyl,
n is a number between 1 and 20,
m is a number between 1 and 5, and
p is a number between 1 and 10.

12. A process according to claim 11 wherein reactive derivatives of the carboxylic acids are their esters, their anhydrides or their acid chlorides, and wherein
R₁ to R₄ are —[CH₂CHR₇O]ₚ—H,
n is 1 to 4,
m is 1 to 3,
p is 1 to 5,
R₅ is a linear or branched C₁₂-C₂₀-alkyl or -alkenyl group,
R₆ is a C₂₄-C₃₆-alkylene group, and
R₇ is H or methyl.

13. A process according to claim 11 wherein
R₁ to R₄ are —[CH₂CHR₇O]ₚ—H,
n is 2,
m is 1,
p is 1 to 3,
R₅ is a linear or branched C₁₂-C₂₀-alkenyl group, and
R₇ is H.

14. A process according to claim 11 wherein the molar ratio of the carboxylic acid to the alkanolamine is between 0.5 and 3.5.

15. A process according to claim 11 wherein the at least one fatty acid ester compound is partially quaternised with an alkylating agent.

16. A process according to claim 15 wherein the at least one fatty acid ester compound is partially quaternised with methyl chloride, benzyl chloride, dimethyl sulfate or diethyl sulphate, preferably with dimethyl sulfate.

17. A process according to claim 11 wherein the at least one fatty acid ester compound is a pre-prepared dispersion in water or is in a blend with a hydroxy compound as an additional additive.

18. A process according to claim 11, wherein R₆ is a substituted or unsaturated C₁-C₃₆-alkylene group.

19. A process according to claim 11 wherein the molar ratio of the carboxylic acid to the alkanolamine is between 1.0 and 3.5.

20. A process according to claim 11 wherein the molar ratio of the carboxylic acid to the alkanolamine is between 2.5 and 3.5.

21. A process for softening, debonding, or bulking pulp and paper comprising the step of mixing at least one fatty acid ester compound wherein the at least one fatty acid ester compound is a product of an esterification reaction of an alkanolamine of general formula (I) 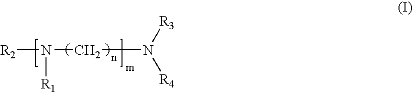
with a carboxylic acid or a reactive derivative thereof, of general formula
R₅COOH,
or with a dicarboxylic acid or a reactive derivative thereof, of general formula
HOOC—R₆—COOH,
wherein at least one of the radicals R₁ to R₄ is —[CH₂CHR₇O]ₚ—H, the others being H or C₁-C₆ alkyl,
R₅ is a linear or branched C₂-C₂₂-alkyl or -alkenyl group,
R₆ is a C₁-C₃₆-alkylene group, or an arylene group,
R₇ is H or C₁-C₄-alkyl,
n is a number between 1 and 20,
m is a number between 1 and 5, and
p is a number between 1 and 10,
with a pulp slurry and subsequently forming a paper sheet.

22. A paper comprising the pulp produced by the process according to claim 1.

23. A paper produced by the process according to claim 11.

24. A paper produced by the process according to claim 21.
